(19) United States
(12) Patent Application Publication
(10) Pub. No.: US 2025/0134345 A1
(43) Pub. Date: May 1, 2025

(54) ENDOSCOPE SYSTEM AND METHOD OF OPERATING THE SAME

(71) Applicant: FUJIFILM Corporation, Tokyo (JP)

(72) Inventor: Takahiro MORI, Kanagawa (JP)

(73) Assignee: FUJIFILM Corporation, Tokyo (JP)

(21) Appl. No.: 18/929,542

(22) Filed: Oct. 28, 2024

(30) Foreign Application Priority Data
Oct. 30, 2023 (JP) 2023-185507

(51) Int. Cl. A61B 1/00; A61B 1/045; A61B 1/06

(52) U.S. Cl. CPC A61B 1/00009; A61B 1/00043; A61B 1/045; A61B 1/0661

(57) ABSTRACT

An endoscope system according to an exemplary embodiment of the invention consecutively images, with an endoscope, a subject by switching between a reference focus position where a focus position is a preset position and a moved focus position where a position of a focus lens is moved from the reference focus position, performs image analysis on a focus position movement image captured at the moved focus position to recognize a region of interest from the subject, and displays an endoscope image captured at the reference focus position on a screen and notifies of a result of the image analysis.

### CROSS-REFERENCE TO RELATED APPLICATIONS

This application claims priority under 35 U.S.C § 119(a) to Japanese Patent Application No. 2023-185507 filed on 30 Oct. 2023. The above application is hereby expressly incorporated by reference, in its entirety, into the present application.

### BACKGROUND OF THE INVENTION

### 1. Field of the Invention

The present invention relates to an endoscope system and a method of operating the same.

### 2. Description of the Related Art

In recent years, in a medical field, diagnosis and the like using an endoscope system that acquires an image and performs observation have become widespread, and it is common for a doctor to perform observation while manually changing a position of a camera in order to recognize a region of interest such as a lesion. The doctor endeavors to constantly detect all the regions of interest to be observed in an organ.

Accuracy of recognizing the region of interest in endoscopic observation is affected by an experience and skill of the doctor, as well as a degree of fatigue of the doctor. In order to reduce such a variation in diagnostic accuracy by the doctors, a technique for analyzing a vast amount of endoscopic data acquired in daily medical practice using a computer to extract information useful for diagnosis is being developed. For example, a recognition support function in which the computer automatically detects the region of interest during the endoscopic observation is expected to prevent the doctor from overlooking a detection target.

Specifically, an endoscope system of WO2017/81976A (corresponding to US2018/249900A1) has a technique for performing recognition support of a feature region based on a predetermined feature quantity of an observation image. In addition, in a case in which the feature region is continuously recognized in observation images that are sequentially input, a position corresponding to the feature region is highlighted.

### SUMMARY OF THE INVENTION

The feature region recognition of WO2017/81976A is premised on the fact that the region of interest is clearly reflected in an image. However, depending on a shape of a subject, in a range outside a user's viewpoint, such as an edge of the image, a camera may not be in focus, resulting in a blurred image, or the image may have excessive or insufficient brightness. In image analysis of these images, analysis accuracy tends to decrease. Ensuring that the focus and brightness are always optimal during observation relies on the skill of the user, thereby making it difficult to maintain the stability of the analysis accuracy.

An object of the present invention is to provide an endoscope system and a method of operating the same that provide imaging with optimal focus and brightness even though a region of interest is in a range outside a user's viewpoint.

An endoscope system according to an exemplary embodiment of the invention comprises: an endoscope that images a subject; and a processor, in which the endoscope consecutively images the subject by switching between a reference focus position where a focus position is a preset position and a moved focus position where the focus position is moved from the reference focus position, and the processor acquires an endoscope image captured by the endoscope at the reference focus position and a focus position movement image captured by the endoscope at the moved focus position, performs image analysis on the focus position movement image to recognize a region of interest from the subject, and displays the endoscope image on a screen and notifies of a result of the image analysis.

It is preferable that the endoscope captures, as the focus position movement image, at least one of a near point image in which the focus position is at a near point closer to the endoscope than in the endoscope image or a far point image in which the focus position is at a far point farther from the endoscope than in the endoscope image.

It is preferable that the processor measures a deviation amount of the focus position with respect to an in-focus position of the subject using a phase difference detection imaging element provided in the endoscope, and acquires at least one of position information of the near point or position information of the far point.

It is preferable that the processor measures a movement amount of a distal end part of the endoscope, compares the movement amount with a predetermined reference range for the movement amount, reduces an imaging frequency of the focus position movement image with respect to the endoscope image in a case in which the movement amount is a value larger than the reference range, and increases the imaging frequency of the focus position movement image with respect to the endoscope image in a case in which the movement amount is a value smaller than the reference range.

It is preferable that in a case in which the movement amount is a value included in the reference range, the endoscope fixes the imaging frequency of the focus position movement image with respect to the endoscope image and performs imaging.

It is preferable that the endoscope captures a recognition-time focus position image based on the focus position at which the region of interest is recognized, at a higher imaging frequency than the endoscope image, and the processor performs the image analysis on the recognition-time focus position image.

It is preferable that the processor displays the captured recognition-time focus position image on a screen.

It is preferable that the processor performs the image analysis on the endoscope image, and decides the recognition-time focus position image according to an analysis result of the endoscope image.

It is preferable that the endoscope repeats an operation of capturing the endoscope image during a first period spanning a plurality of frames and capturing the focus position movement image during a second period spanning at least one frame.

It is preferable that the processor controls an exposure amount of illumination light, which is emitted from a light source device connected to the endoscope and with which the subject is illuminated, according to the focus position.

A method of operating an endoscope system according to an exemplary embodiment of the invention comprises: a step of, via an endoscope, consecutively imaging a subject by switching between a reference focus position, which is a preset focus position, and a moved focus position, which is the focus position moved from the reference focus position; a step of, via a processor, acquiring an endoscope image captured by the endoscope at the reference focus position and a focus position movement image captured by the endoscope at the moved focus position; a step of, via the processor, performing image analysis on the focus position movement image to recognize a region of interest from the subject; and a step of, via the processor, displaying the endoscope image on a screen and notifying of a result of the image analysis.

According to the exemplary embodiment of the invention, it is possible to achieve imaging with optimal focus and brightness even though a region of interest is in a range outside a user's viewpoint.

### BRIEF DESCRIPTION OF THE DRAWINGS

FIG. 1 is a schematic view of an endoscope system.

FIG. 2 is a block diagram showing a function of the endoscope system.

FIG. 3 is an explanatory diagram of an imaging element having phase difference detection pixels.

FIG. 4 is an explanatory diagram illustrating imaging of a subject having a protruding part.

FIG. 5 is an explanatory diagram illustrating imaging of the subject having a depressed part.

FIG. 6 is an explanatory diagram illustrating imaging of the subject that is a tubular digestive tract.

FIG. 7 is an explanatory diagram of an endoscope image, a near point image, and a far point image that are captured in a special observation mode.

FIG. 8 is an explanatory diagram illustrating imaging of the subject using a pre-recognition imaging pattern.

FIG. 9 is an explanatory diagram illustrating imaging of the subject with a movement amount larger than a reference range in the pre-recognition imaging pattern.

FIG. 10 is an explanatory diagram illustrating imaging of the subject with a movement amount smaller than a reference range in the pre-recognition imaging pattern.

FIG. 11 is an explanatory diagram illustrating imaging of the subject by switching to a first post-recognition imaging pattern.

FIG. 12 is an explanatory diagram illustrating imaging of the subject by switching to a second post-recognition imaging pattern.

FIG. 13 is an explanatory diagram illustrating imaging of the subject by switching to a third post-recognition imaging pattern.

FIG. 14 is a flowchart showing a series of steps for controlling capturing of a focus position movement image in an exemplary embodiment of the invention.

FIG. 15 is an explanatory diagram illustrating image analysis on an image captured at a focus position set by a user in a second embodiment.

### DESCRIPTION OF THE PREFERRED EMBODIMENTS

### First Embodiment

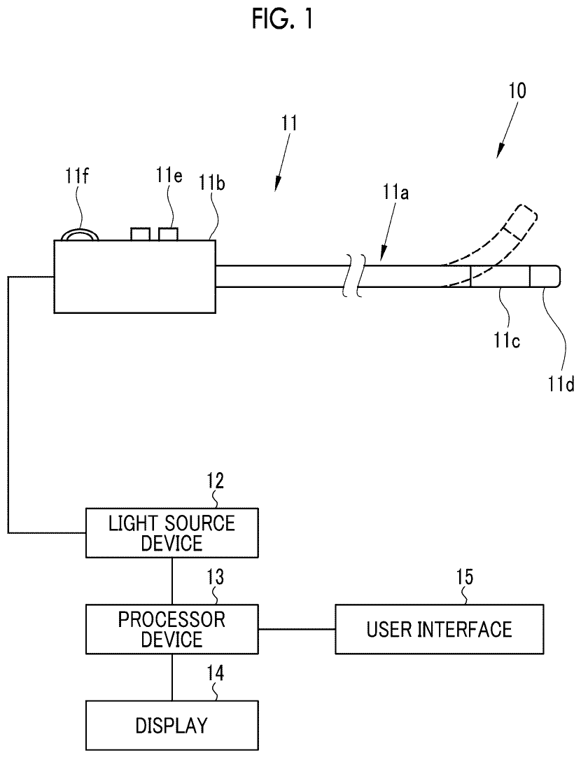

As shown in FIG. 1, an endoscope system 10 according to a first embodiment of the invention includes an endoscope 11, a light source device 12, a processor device 13, a display 14, and a user interface (UI) 15. The endoscope 11 is optically connected to the light source device 12 and electrically connected to the processor device 13. The light source device 12 supplies illumination light to the endoscope 11.

The endoscope 11 illuminates a subject with illumination light and acquires an endoscope image by imaging the subject. The endoscope 11 has an insertion part 11a that is to be inserted into a living body (inside an object to be examined) having the subject, and an operation part 11b that is provided at a base end portion of the insertion part 11a. A bendable part 11c and a distal end part 11d are provided on a distal end side of the insertion part 11a. The bendable part 11c is operated by the operation part 11b to be bent in a desired direction. The distal end part 11d irradiates the subject with illumination light and receives reflected light from the subject to image the subject. The operation part 11b is provided with a mode selector switch 11e that is used for an operation for switching a mode and a zoom operation portion 11f that is used for a zoom operation.

The processor device 13 is electrically connected to the display 14 and the user interface 15. The processor device 13 receives an image signal from the endoscope 11, and performs various kinds of processing based on the image signal. An external recording unit (not shown), which records an image, image information, and the like, may be connected to the processor device 13. The display 14 outputs and displays a captured image of the subject, image information, and the like, which have been image-processed by the processor device 13. The user interface 15 includes a keyboard, a mouse, a touch pad, a microphone, a foot pedal, and the like, and has a function of receiving an input operation such as function setting.

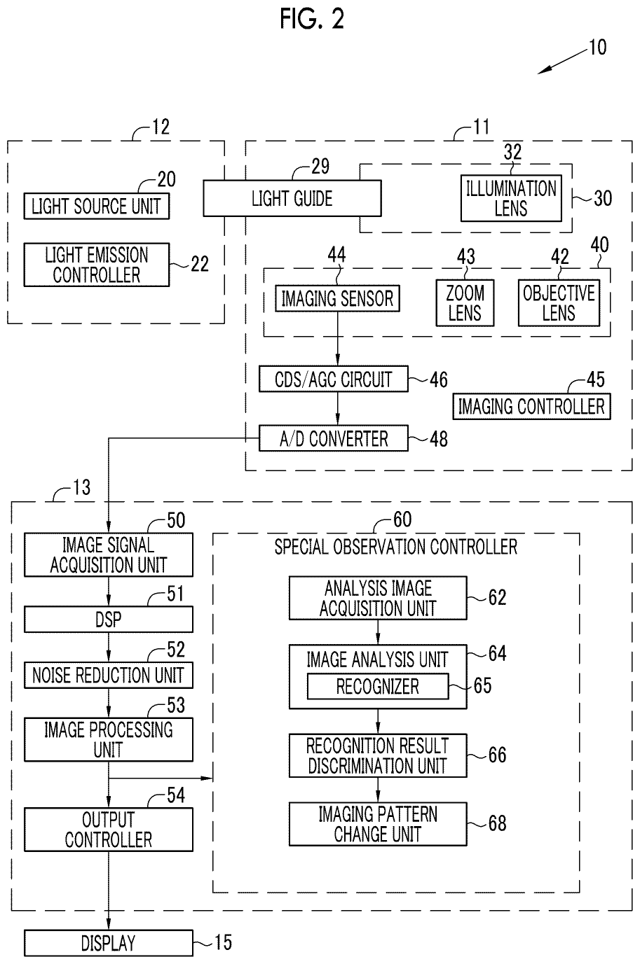

As shown in FIG. 2, in the endoscope system 10, the light source device 12 propagates emitted illumination light to the endoscope 11 via a light guide 29, the endoscope 11 transmits an image signal imaged using illumination light to the processor device 13, and the processor device 13 generates an image to be displayed on the display 14 and performs image analysis.

The light source device 12 comprises a light source unit 20 that emits one or a plurality of illumination light beams, and a light emission controller 22 that generates a drive current (drive signal) for controlling a light emission timing, a light emission amount, and the like of the light source unit 20 and that supplies the drive current to the light source unit 20 to cause the light source unit 20 to emit light.

The function of the light emission controller 22 is realized by a light source control processor (not shown) included in the light source device 12, and the light emission controller 22 supplies a drive current (drive signal) for controlling the light emission timing, the amount of illumination light, and the like to the light source unit 20 to control the illumination light to be emitted in response to reception of a light emission control signal. In a case in which the light source device 12 and the processor device 13 are electrically connected, the function of the light source control processor may be realized by a central controller (not shown) instead of the light source control processor.

The light emitted from the light source unit 20 is incident into the light guide 29. The light guide 29 is built into the endoscope 11 and a universal cord. The universal cord is a cord that connects the endoscope 11 to the light source device 12 and the processor device 13. The light guide 29 propagates the light from the light source unit 20 to the distal end part 11d of the endoscope 11.

The endoscope 11 is provided with an illumination optical system 30 and an imaging optical system 40. The illumination optical system 30 has an illumination lens 32, and the illumination light propagated by the light guide 29 is applied to the subject via the illumination lens 32. The imaging optical system 40 includes an objective lens 42, a zoom lens 43, and an imaging sensor 44. Reflected light of the illumination light returning from the subject irradiated with the illumination light is incident into the imaging sensor 44 via the objective lens 42 and the zoom lens 43. Thereby, an image of the subject is formed on the imaging sensor 44 which is a color imaging sensor. The image formed on the imaging sensor 44 is transmitted to the processor device 13 as an image signal via a correlated double sampling/automatic gain control (CDS/AGC) circuit 46 and an analog/digital (A/D) converter 48.

In the processor device 13, a program related to each processing is incorporated in a program memory (not shown). In a case in which a central controller configured by a processor executes the program in the program memory, functions of an image signal acquisition unit 50, a digital signal processor (DSP) 51, a noise reduction unit 52, an image processing unit 53, an output controller 54, and a special observation controller 60 are realized. The special observation controller 60 comprises an analysis image acquisition unit 62, an image analysis unit 64 including a recognizer 65, a recognition result discrimination unit 66, and an imaging pattern change unit 68.

The zoom lens 43 is a lens group consisting of a plurality of lenses for enlarging or reducing a size of the subject, and includes a focus lens that is movable in an optical axis direction. In near view imaging in which the subject is observed at a close magnified distance, such as in a case of observing a structure of a narrow blood vessel or performing surgical treatment, the focus lens is moved such that a near view is in focus. On the other hand, in distant view imaging in which the subject is observed over a wide range, such as in a case of checking a relatively large lesion, the focus lens is moved such that a distant view is in focus. The depth of field, which is a range in which the subject appears to be in focus, changes due to the control of the zoom lens 43 that moves the focus lens.

The focus lens can be moved either manually by the user operating the zoom operation portion 11f or automatically based on preset setting. In either case, the zoom control is executed on the zoom lens 43 by the imaging controller 45. In the zoom control, the focus lens is freely moved in the optical axis direction, thereby enlarging or reducing the size of the image of the subject formed on the imaging sensor 44.

Since a length of an imaging frame is constant, the imaging sensor 44 is controlled to alternately repeat an accumulation period and a readout period every certain time, for example, every 1/60 seconds. That is, the imaging is performed at a frame rate of 60 frames per second (fps). The shutter speed of the electronic shutter may be changed to adjust the length of the imaging frame.

As the imaging sensor 44, a photoelectric conversion element (imaging element) such as a charge coupled device (CCD) sensor or a complementary metal-oxide semiconductor (CMOS) sensor is used. The imaging sensor 44 performs, for example, an accumulation operation of performing photoelectric conversion of received light and accumulating signal charges corresponding to the amount of received light for each pixel and a readout operation of reading out the accumulated signal charges, within an acquisition period of one frame. The signal charge for each pixel read out from the imaging sensor 44 is converted into a voltage signal and input to the CDS/AGC circuit 46. The light source device 12 generates illumination light in accordance with a timing of the accumulation operation of the imaging sensor 44 and causes the illumination light to be incident into the light guide 29.

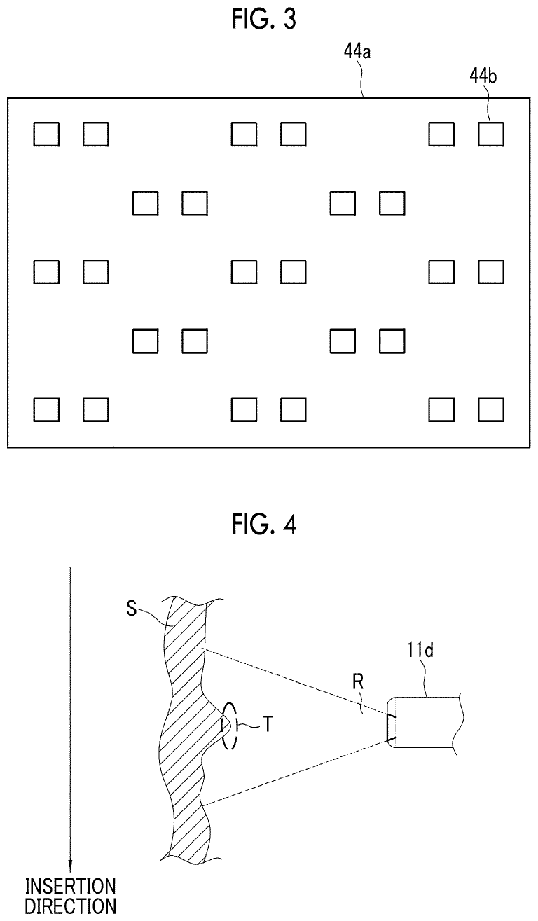

As shown in FIG. 3, the imaging sensor 44 comprises a phase difference detection imaging element 44a, which is an imaging element in which phase difference detection pixels 44b are disposed. The phase difference detection pixels 44b are evenly disposed in the phase difference detection imaging element 44a. By using the phase difference detection pixels 44b, it is possible to specify a focal position (in-focus position) where the subject is in focus by phase difference auto-focusing, which is one of autofocus techniques. For example, in phase difference auto-focusing, it is preferable to use the phase difference detection pixels 44b disposed mainly in a peripheral portion of the phase difference detection imaging element 44a. In addition, it is further preferable to use the phase difference detection pixels 44b disposed only in the peripheral portion of the phase difference detection imaging element 44a.

In endoscopic observation, imaging is performed by moving the focus lens to a focal position specified using a focus target. As for the focal position, the light incident from the objective lens 42 is divided into two by the zoom lens 43, which is a lens group, a difference in the focus target between the two formed images is expressed as a deviation amount, and a position where the subject is in focus is specified based on the deviation amount. In other words, the position of the focus lens where no difference in the subject occurs or where the deviation amount is negligible is the focal position.

Each pixel of the imaging sensor 44 is provided with any of a blue pixel (B pixel) having a blue (B) color filter, a green pixel (G pixel) having a green (G) color filter, or a red pixel (R pixel) having a red (R) color filter. For example, the imaging sensor 44 is preferably a color imaging sensor of a Bayer array in which a ratio of the number of pixels of the B pixels, the G pixels, and the R pixels is 1:2:1.

The B color filter mainly transmits light in a blue band, specifically, light of which a wavelength range is 380 to 560 nm (blue transmission range). A peak wavelength at which a transmittance is maximized exists around 460 to 470 nm. The G color filter mainly transmits light in a green band, specifically, light of which a wavelength range is 450 to 630 nm (green transmission range). The R color filter mainly transmits light in a red band, specifically, light of which a wavelength range is 580 to 760 nm (red transmission range).

In addition, a complementary color imaging sensor comprising complementary color filters corresponding to cyan (C), magenta (M), yellow (Y), and green (G) may be used instead of the primary color imaging sensor 44. In a case in which the complementary color imaging sensor is used, image signals corresponding to four colors of C, M, Y, and G are output. Therefore, in a case in which the image signals corresponding to four colors of C, M, Y, and G are converted into image signals corresponding to three colors of R, G, and B by complementary color-primary color conversion, image signals corresponding to the same respective colors of R, G, and B as those of the imaging sensor 44 can be obtained.

The imaging controller 45 drives and controls the imaging sensor 44 in response to the mode selector switch 11e, the user interface 15 via the processor device 13, and an instruction from the imaging pattern change unit 68 described below to control imaging according to each observation mode. In the control of the imaging, adjustment of an exposure period through setting of a shutter speed of an electronic shutter (not shown) of the imaging sensor 44 is performed. In a case in which an imaging pattern is switched, the illumination light emitted via the light emission controller 22 is switched. In addition, the imaging controller 45 generates a control signal for controlling the light emission timing and the amount of light emission of the light source unit 20 based on a focus position described below, and transmits a drive current (drive signal) corresponding to the control signal to each light source in the light emission controller 22 to emit light.

The imaging controller 45 has a function of adjusting the focus position, and further has a function of changing the focus position by an optional moving amount from the focus position decided at the time of imaging, in addition to a function of adjusting the focus position by operating the zoom operation portion 11f (manual focusing) and a function of automatically adjusting the focus position on a subject S by the processor device 13 (auto focusing). The decided focus position includes a focus position that has not been changed since the start of the imaging.

The CDS/AGC circuit 46 performs correlated double sampling (CDS) or automatic gain control (AGC) on the analog image signals obtained from the imaging sensor 44. The image signal that has passed through the CDS/AGC circuit 46 is converted into a digital image signal by the A/D converter 48. The digital image signal after the A/D conversion is input to the processor device 13.

The image signal acquisition unit 50 receives an image signal input from the endoscope 11 and transmits the received image signal to the DSP 51. The output controller 54 transmits an image signal of an image to be displayed, which has been processed by the DSP 51, the noise reduction unit 52, and the image processing unit 53, to the display 14.

The DSP 51 performs various kinds of signal processing, such as defect correction processing, offset processing, gain correction processing, linear matrix processing, gamma conversion processing, demosaicing, and YC conversion processing, on the image signal received by the image signal acquisition unit 50. In the defect correction processing, a signal of a defective pixel of the imaging sensor 44 is corrected. In the offset processing, a dark current component is removed from the image signal that has passed through the defect correction processing, and an accurate zero level is set. In the gain correction processing, a signal level of each image signal is adjusted by multiplying the image signal of each color after the offset processing by a specific gain. The image signal of each color after the gain correction processing is subjected to the linear matrix processing for enhancing color reproducibility.

After that, brightness and chroma saturation of each image signal are adjusted by the gamma conversion processing. The image signal after the linear matrix processing is subjected to the demosaicing (also referred to as isotropic processing or synchronization processing), and a signal of a color missing from each pixel is generated by interpolation. By the demosaicing, all pixels have signals of respective colors of R, G, and B. The DSP 51 performs the YC conversion processing on each image signal after the demosaicing, and outputs a brightness signal Y, a color difference signal Cb, and a color difference signal Cr to the noise reduction unit 52.

The noise reduction unit 52 performs noise reduction processing by, for example, a moving average method or a median filter method on the image signal that has passed through the demosaicing or the like by the DSP 51. The image signal with reduced noise is input to the image processing unit 53.

The image processing unit 53 further performs color conversion processing, such as 3×3 matrix processing, gradation transformation processing, and three-dimensional look up table (LUT) processing, on the input image signal for one frame. Then, various kinds of color enhancement processing are performed on the RGB image data that has been subjected to the color conversion processing. Structure enhancement processing such as spatial frequency enhancement is performed on the RGB image data that has been subjected to the color enhancement processing. In the image processing, the RGB image data that has passed through the structure enhancement processing is input to the output controller 54 as an endoscope image.

The output controller 54 sequentially acquires the endoscope images from the image processing unit 53, and converts the acquired endoscope images into video signals that enable full-color display on the display 14. The converted video signal is output to and displayed on the display 14. Accordingly, a doctor or the like can observe the subject by using a still image or a video image of the endoscope image.

The analysis image acquisition unit 62 acquires an image for region-of-interest recognition from among images captured in a special observation mode. The image to be acquired is obtained from the image processing unit 53 in accordance with an imaging pattern in the special observation mode.

The image analysis unit 64 performs image analysis on the analysis image received from the analysis image acquisition unit 62 to recognize the region of interest. The recognizer 65, which is a trained model optimized for recognition of a region of interest of an imaging part, is used for the recognition of the region of interest. In addition, a plurality of the recognizers 65 corresponding to different parts of the subject may be provided in order to perform recognition with higher accuracy. In this case, the image analysis unit 64 discriminates the imaging part and inputs an image for region-of-interest recognition to the recognizer 65 corresponding to the part.

The recognizer 65 comprises a convolutional neural network (CNN), which is a computer algorithm consisting of a neural network that performs machine learning, and recognizes a region of interest captured in the input endoscope image according to the pre-implemented learning content regarding the region of interest in the object to be examined. The recognition of the region of interest includes detection of a lesion, determination of a lesion, specification of an organ, measurement of a lesion, and the like. In addition, it is preferable to calculate a reliability degree and the like in a recognition result. The recognizer 65 has functionality of a trained deep learning model that has been trained using a large amount of data set. The large amount of data set includes images including a region of interest at each observation site and images not including the region of interest, where the region of interest is a lesion, a characteristic structure in each organ, or the like.

The learning in the recognizer 65 uses a data set including many images of the imaging part corresponding to the endoscopic observation as the subject. The recognizer 65 is used in a sufficiently trained state for the images of the imaging part corresponding to a type of endoscope 11 to be used. In a case in which the endoscope 11 is an upper gastrointestinal endoscope, imaging parts such as “esophagus”, “stomach”, and “duodenum” are the subject. In a case in which the endoscope 11 is a lower gastrointestinal endoscope, imaging parts such as “rectum”, “large intestine”, and “small intestine” are the subject. The image analysis unit 64 may classify the parts of the image for the region-of-interest recognition and may properly use a plurality of the recognizers 65 trained for each part according to a classification result.

The recognition result discrimination unit 66 acquires at least information on the presence or absence of the region of interest as the recognition result of the recognizer 65. In addition, the recognition result discrimination unit 66 also has a function of discriminating whether images that have been subjected to consecutive recognition processing or images captured within a certain period have the same region of interest. The discriminated recognition result is transmitted to the imaging pattern change unit 68 together with the focus position information. In addition, in a case in which the region of interest is recognized at a plurality of focus positions, the recognition result of the focus position where the subject is in focus is discriminated using the reliability degree calculated by the recognizer 65.

The imaging pattern change unit 68 issues an imaging pattern change instruction in the special observation mode according to the discrimination result of the recognition result discrimination unit 66. In a case in which it is discriminated that the region of interest is present in consecutive imaging, the imaging pattern is changed based on a recognition-time focus position image, which is an image in which the region of interest is recognized. That is, the imaging pattern in the special observation mode includes a pre-recognition imaging pattern before the region of interest is recognized, and a post-recognition imaging pattern after the region of interest is recognized. The imaging pattern change instruction is transmitted to the imaging controller 45 to change the imaging pattern. The imaging pattern may be changed at a timing at which the region of interest is consecutively discriminated to be the same, instead of recognizing the region of interest once.

The endoscope system 10 according to the present embodiment has a function of performing observation by optionally switching between a normal observation mode in which image analysis is not performed and a special observation mode in which image analysis is performed. In the normal observation mode, the subject is imaged and observed based on the focus position set manually or automatically by endoscope 11.

In the special observation mode, imaging is performed by switching between frames of endoscope images captured at a reference focus position, which is a focus position set in the same manner as in the normal observation mode, and frames of focus position movement images captured at a moved focus position, which is a position moved from the reference focus position. The focus position movement image is used for image analysis. The observation in the special observation mode involves imaging in which the focus position is controlled to switch in units of one frame. There are a plurality of patterns for capturing the normal images and the focus position movement images in the special observation mode, and each pattern is switched according to the user's operation or the recognition result.

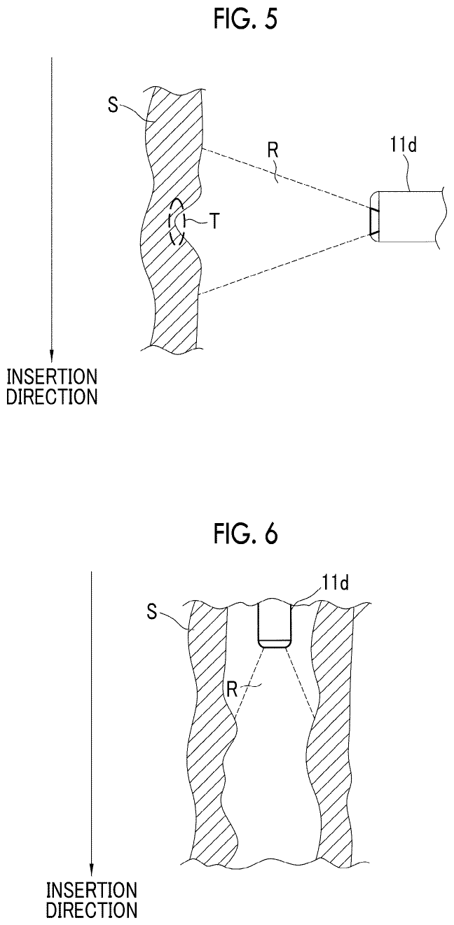

As shown in FIGS. 4 and 5, in the normal observation mode, even though the user focuses on a focus target T being observed, the region of interest may be out of focus or the exposure amount may be inappropriate. For example, in a case in which the focus position is adjusted to the focus target T that is a protruding part or a depressed part of the subject S, it is difficult to focus on a part with a relatively flat shape.

FIG. 4 is an example in which the focus target T, which is a protruding part of the subject S, is in focus. The focus position and the exposure amount are adjusted for the protruding part, which is the focus target T, and the focus target T is in focus and imaging is performed with brightness suitable for observation. On the other hand, a non-protruding part of the subject S is observed at a farther distance from the objective lens 42 than the protruding part, and therefore may be out of focus and may be insufficient in the exposure amount.

FIG. 5 is an example in which the focus target T, which is a depressed part of the subject S, is in focus. The focus position and the exposure amount are adjusted for the depressed part, which is the focus target T, and the focus target T is in focus and imaging is performed with brightness suitable for observation. On the other hand, a non-depressed part of the subject S is observed at a closer distance to the objective lens 42 than the depressed part, and therefore may be out of focus and may be excessive in the exposure amount.

FIG. 6 is a cross-sectional view of a tubular digestive tract in a case in which endoscopic observation is performed on the tubular digestive tract, and observation is performed while the distal end part 11d of the endoscope 11 advances in an insertion direction in the tubular digestive tract. The tubular digestive tract is, for example, the esophagus. As shown in FIG. 6, in addition to a clear protruding part or depressed part, there may be a plurality of options for the shape of the subject S and the focus position at which the subject S is imaged. In particular, in a case in which the user makes observation by minimizing the movement of the distal end part 11d, it is required to appropriately set the focus position and the exposure amount and to perform the region-of-interest recognition processing with high accuracy.

In the special observation mode, in a case in which the focal position to be specified in the same manner as in the normal observation mode is specified by using a deviation amount, a deviation amount of the focus position with respect to the focal position of the subject S is measured by using the phase difference detection imaging element 44a, and at least one of position information of a near point or position information of a far point is acquired according to the deviation amount. A near point image is captured using the position information of the near point, and a far point image is captured using the position information of the far point. It is discriminated whether the focal position is at a near point or at a far point with respect to the distal end part 11d of the endoscope 11 as compared with the focus position before the phase difference auto-focusing is performed.

Further, the exposure amount is controlled in accordance with each mode and each pattern, and the turning on or off of each light source and the amount of light emitted in a case in which the light source is turned on are independently controlled by the light emission controller 22. In the exposure amount control, it is preferable that opening and closing of the shutter is controlled in synchronization with the imaging frame.

In the normal observation mode, imaging is performed by deciding a focus position, which is the position of the focus lens, by manual focusing or auto focusing, and a normal image is generated as a display image. The illumination light remains turned on during the observation, and the exposure period is adjusted by the opening and closing of the shutter or the like.

In the pre-recognition imaging mode of the special observation mode, the subject S is consecutively imaged with the endoscope by switching between the reference focus position where the focus position is a preset position and the moved focus position where the focus position is moved from the reference focus position, in the same manner as in the normal observation mode. An endoscope image 70 captured at the reference focus position and a focus position movement image captured at the moved focus position are acquired by imaging, image analysis is performed on the focus position movement image to recognize the region of interest from the subject S, and the endoscope image 70 is displayed on a screen and notification of a result of the image analysis is performed. The consecutive imaging is performed in an imaging pattern where the endoscope image and the focus position movement image are switched in units of one frame.

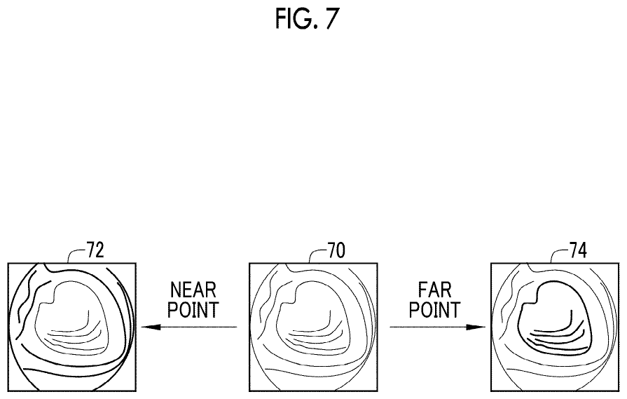

As shown in FIG. 7, the focus position movement image captured by switching from the endoscope image 70 in units of one frame includes a near point image 72 captured at a near point closer to the distal end part 11d than the focus position of the endoscope image 70, and a far point image 74 captured at a far point farther from the distal end part 11d than the focus position of the endoscope image 70. In the special observation mode, imaging is performed while switching between at least one of the near point image 72 or the far point image 74 as the focus position movement image and the endoscope image 70.

In the pre-recognition imaging pattern, the endoscope 11 is moved to an imaging part desired by the user, and the focus position movement images are captured at regular intervals while the endoscope images 70 are consecutively captured. For example, an operation of capturing the endoscope image 70 during a first period spanning a plurality of frames such as three frames and then capturing the focus position movement image during a second period spanning at least one frame is repeated. The second period may be divided into a second A period in which imaging is performed at the near point and a second B period in which imaging is performed at the far point.

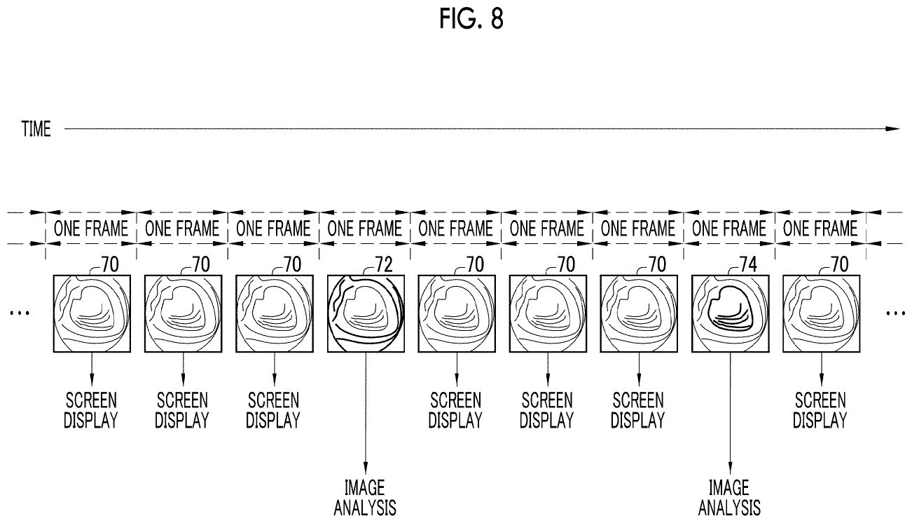

As shown in FIG. 8, in the pre-recognition imaging pattern in which both the near point image 72 and the far point image 74 are captured as the focus position movement images, for example, three frames of the endoscope images 70, one frame of the near point image 72, three frames of the endoscope image 70, and one frame of the far point image 74 are captured repeatedly in this order. The endoscope image 70 is displayed on the screen in the same manner as in the normal observation mode, and the near point image 72 and the far point image 74 are subjected to image analysis.

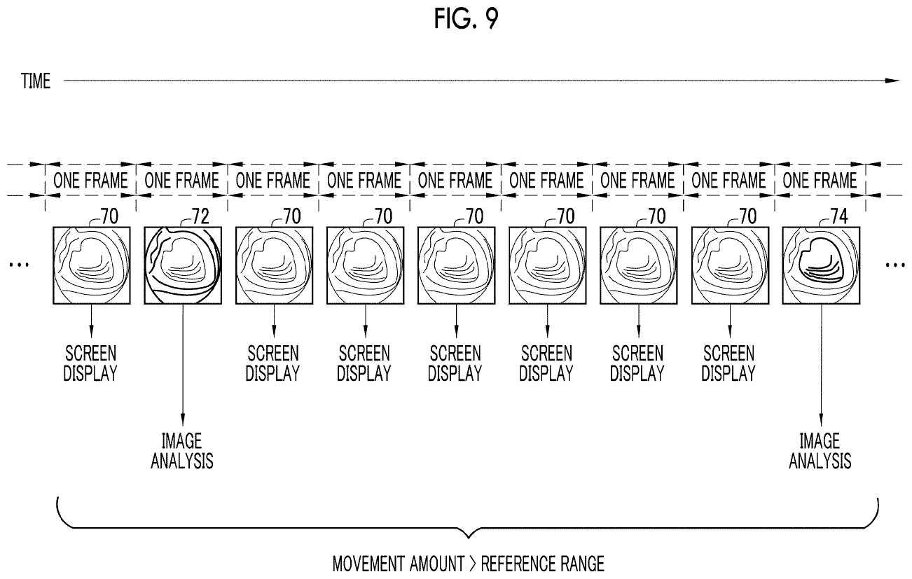

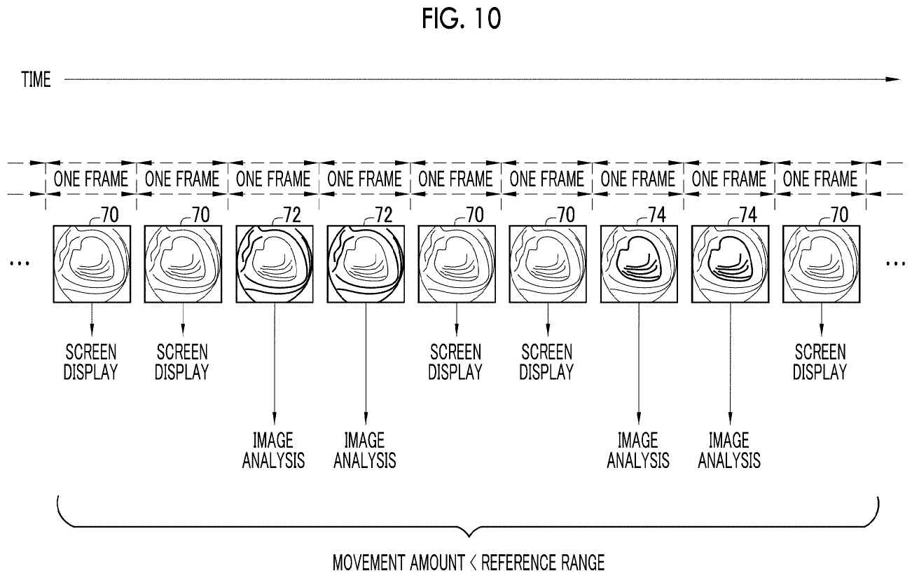

As shown in FIGS. 9 and 10, in the pre-recognition imaging pattern, an imaging ratio of the endoscope image 70 to the focus position movement image, that is, a ratio of the first period to the second period may be automatically changed based on the movement amount of the endoscope 11. In a case of measuring the movement amount, the processor device 13 measures the movement amount of the distal end part 11d of the endoscope 11 and compares the movement amount with a predetermined reference range for the movement amount. It is preferable that the reference range is decided based on an average moving speed of the endoscope 11 in endoscopic observation, or the like.

As shown in FIG. 9, as a result of the comparison, in a case in which the movement amount is a value larger than the reference range, the imaging frequency of the focus position movement image with respect to the endoscope image 70 is reduced. For example, with an imaging ratio of 6:1 for the endoscope image 70 and the focus position movement image, imaging is repeatedly performed in which six frames of the endoscope images 70, one frame of the near point image 72, six frames of the endoscope images 70, and one frame of the far point image 74 are switched in this order. In a case in which the movement amount is large, that is, in a case in which the user moves the insertion part 11a at a high speed, it is possible to grasp the imaging part intensively, for example, while changing an imaging area. In addition, by continuing the image analysis, overlooking during movement can be prevented. In addition, a threshold value may be set for the movement amount, and the imaging frequency of the focus position movement image may be gradually reduced as the movement amount increases.

As shown in FIG. 10, as a result of the comparison, in a case in which the movement amount is a value smaller than the reference range, the imaging frequency of the focus position movement image with respect to the endoscope image 70 is increased. For example, with an imaging ratio of 1:1 for the endoscope image 70 and the focus position movement image, imaging is repeatedly performed in which two frames of the endoscope images 70, two frames of the near point images 72, two frames of the endoscope images 70, and two frame of the far point images 74 are switched in this order. In a case in which the movement amount is small, that is, in a case in which the user moves the insertion part 11a slowly, accurate analysis results can be obtained by performing image analysis on a large number of images.

It is preferable that the imaging frequency of the focus position movement image is set to a frequency at which the observation of the endoscope image 70 by the screen display is not affected, for example, the screen display of the endoscope image 70 is maintained at 30 fps or more. In this case, in a case in which the screen display maintains a frame rate of 30 fps, the imaging frequency of the focus position movement image may be set higher than that of the endoscope image 70. In a case in which the imaging at the moved focus position is performed in a plurality of consecutive frames, the imaging may be performed by further moving the focus position. For example, in a case of capturing three consecutive frames of the near point images 72, a first near point image, a second near point image, and a third near point image having different focus positions are captured.

It is preferable that in a case in which the movement amount is a value included in the reference range, the imaging frequency of the focus position movement image with respect to the endoscope image 70 is fixed to perform imaging. The fixed imaging frequency of the focus position movement image is set higher than in a case in which the movement amount is larger than the reference range and lower than in a case in which the movement amount is smaller than the reference range. For example, with an imaging ratio of 3:1 for the endoscope image 70 and the focus position movement image, the switching of capturing one frame of the focus position movement image for every six frames of capturing the endoscope image 70 (see FIG. 8) is performed in a fixed manner.

The movement amount can be obtained by measuring the bending operation or zoom operation using the operation part 11b or the insertion length of the insertion part 11a and calculating the moving amount of the insertion part 11a of the endoscope 11, or by comparing the analysis results of images with the same focus position.

In a case in which image analysis is used, for example, feature points are discriminated in the image analysis of the near point image 72, and compared with feature points of the near point image 72 acquired before and after the discrimination. An accurate moving amount may be calculated using an imaging magnification or an observation distance to measure the movement amount, or a relative movement amount based on a change in the position of the image may be measured. In addition, the same discrimination is performed on the far point image 74. The movement amount may be measured by inputting the endoscope image 70 to the recognizer 65. In this case, the recognizer 65 is implemented in a manner that does not interfere with the image display.

In a case in which the region of interest is recognized in the plurality of focus position movement images with different focus positions in the image analysis of the pre-recognition imaging pattern, the recognizer 65 discriminates the focus position movement image, which is an accurate recognition result, by using the reliability degree calculated by the recognizer 65. There is a method of recognizing a region of interest by detecting a feature quantity in an image, but even though the feature quantity is sufficient, in a state in which it can be discriminated that the focus position is inappropriate, such as in a case in which an outline is unclear and cannot be specified, learning is performed to calculate a low reliability degree.

In the post-recognition imaging pattern, the recognition-time focus position image based on the focus position at which the region of interest is recognized in the image analysis of the pre-recognition imaging pattern is consecutively captured or is captured at a higher imaging frequency than the endoscope image 70. The post-recognition imaging pattern is in a state in which the imaging frequency of the recognition-time focus position image is higher than that of the focus position movement image in the pre-recognition imaging pattern in which the movement amount is the same. By capturing the analysis image in a concentrated manner, it is possible to achieve higher accuracy in image analysis.

The post-recognition imaging pattern may be properly used according to the application of the focus position movement image. For example, a first post-recognition imaging pattern for performing image analysis, a second post-recognition imaging pattern for performing screen display, and a third post-recognition imaging pattern for performing both screen display and image analysis are used properly. In the selection of the post-recognition imaging pattern, the setting of the post-recognition imaging pattern is changed during the endoscopic observation by consecutive pressing of the mode selector switch 11e or the like.

In the post-recognition imaging pattern, the frame rate in the screen display of the endoscope image 70 is displayed lower than the frame rate during imaging. For example, in a post-recognition imaging pattern in which imaging is performed at 60 fps, in a case in which an imaging ratio is 1:1 for the endoscope image 70 and the focus position movement image, the screen display of the endoscope image 70 is adjusted to be an evenly spaced 30 fps.

In a case in which the focus position movement image is displayed on the screen, the focus position movement image may be displayed in a divided manner on the display 14. For example, the endoscope image 70 is displayed on a main screen, and the focus position movement image is displayed on a sub screen. The focus position movement image displayed on the sub screen may be a still image or may be displayed as a moving image in a case in which the focus position movement image is captured at a sufficient frame rate, such as 30 fps or more. In addition, instead of the screen division, a display (not shown) different from display 14 may be electrically connected to the processor device 13, and the endoscope image 70 and the focus position movement image may be displayed on different screens.

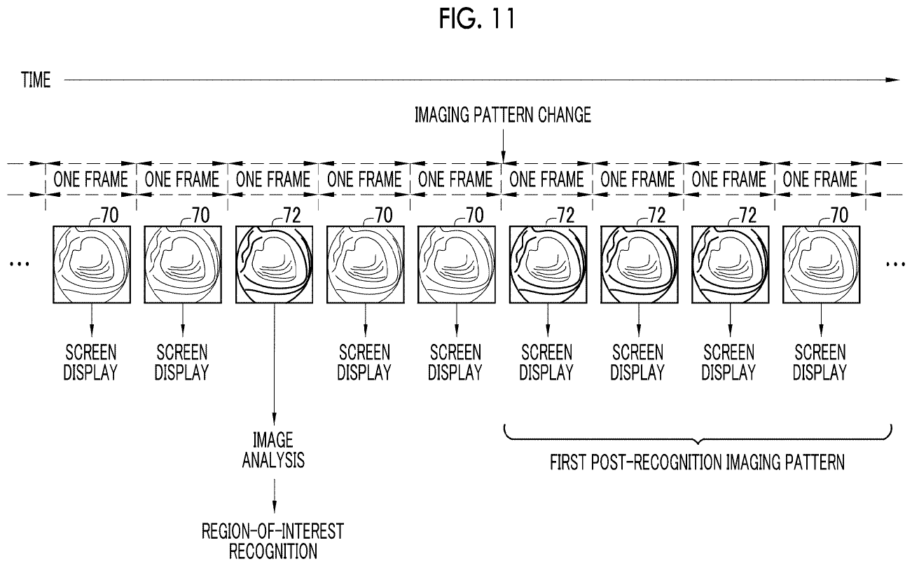

As shown in FIG. 11, in the first post-recognition imaging pattern, the recognition-time focus position image based on the focus position at which the region of interest is recognized is consecutively captured at a higher imaging frequency than the endoscope image 70 and image analysis is performed. In a case in which the recognition-time focus position image is the near point image 72, the near point image is consecutively captured and image analysis is performed.

In switching from the image analysis to the post-recognition imaging pattern, the image capturing of the pre-recognition imaging pattern continues according to the image size of the focus position movement image in the pre-recognition imaging pattern, the performance of the recognizer 65, the frame rate setting, and the like, but the imaging controller 45 switches to the post-recognition imaging pattern according to the imaging pattern change instruction from the imaging pattern change unit 68.

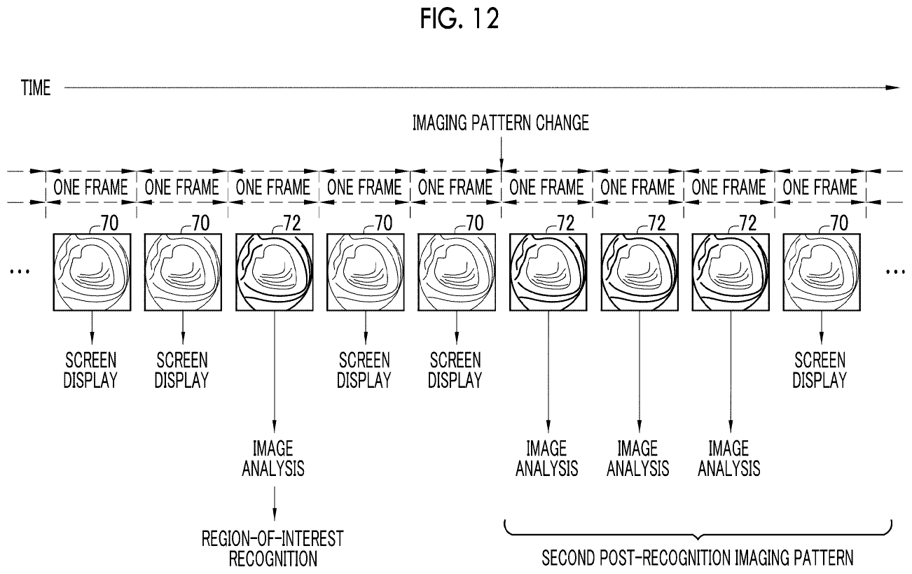

As shown in FIG. 12, in the second post-recognition imaging pattern, the captured recognition-time focus position image is displayed on the screen. In a case in which the focus position movement image in which the region of interest is recognized is the near point image 72, the near point image 72 is consecutively displayed. Since a state in which the imaging is switched from the endoscope image 70 at short intervals is inappropriate for observation, the recognition-time focus position image is consecutively captured for a sufficient period, for example, 0.5 seconds or longer, and displayed on the screen.

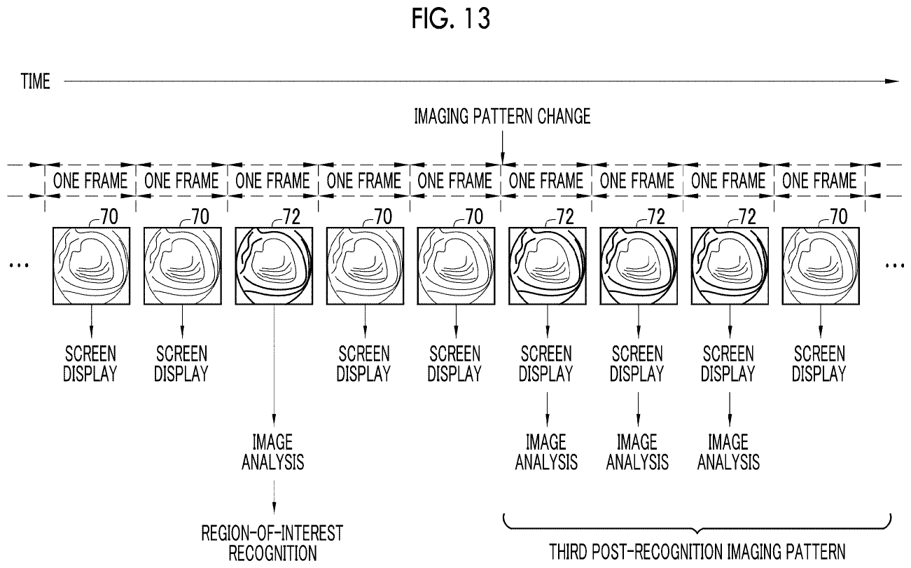

As shown in FIG. 13, in the third post-recognition imaging pattern, both the screen display and the image analysis are performed on the captured recognition-time focus position image. The screen display may be executed by the sub screen display as in the second post-recognition imaging pattern, or the image analysis is executed and then the sub screen display of the focus position movement image as the moving image or the still image together with the result of the image analysis may be executed.

In the first post-recognition imaging pattern and the third post-recognition imaging pattern in which image analysis is performed, in a case in which the region of interest is detected, a notification is performed. The notification is performed by a voice, a warning sound, or by highlighting on the display screen. The output controller 54 controls the notification content based on the recognition result discriminated by the recognition result discrimination unit 66.

Since the post-recognition imaging pattern is used to capture the analysis image of the region of interest, the imaging pattern is automatically switched to the pre-recognition imaging pattern in a case in which the imaging range is away from the region of interest or in a case in which a predetermined period of time has elapsed. By automatically switching to the pre-recognition imaging pattern, it is possible to perform observation and image analysis with a focus position suitable for different imaging ranges.

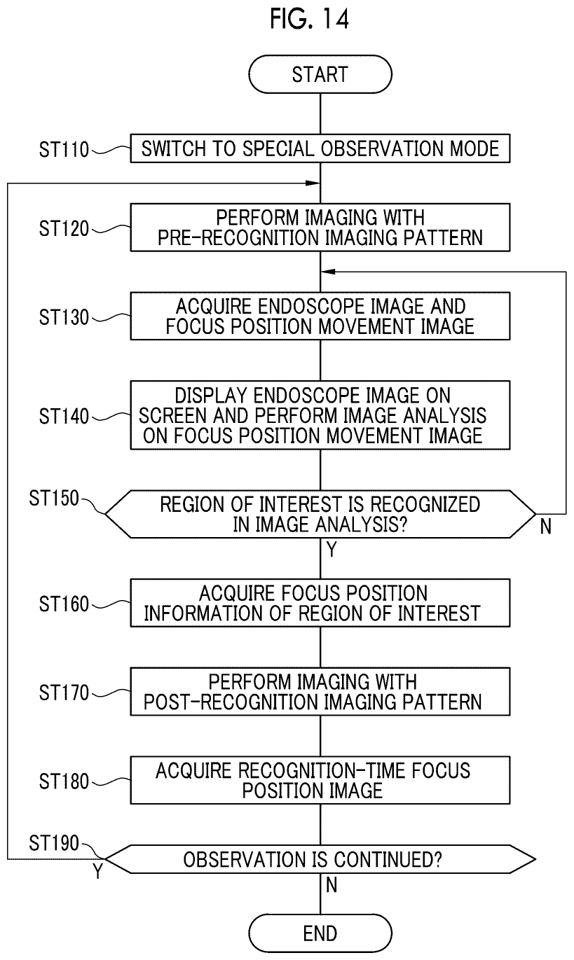

A series of steps for controlling the capturing of the focus position movement image in an exemplary embodiment of the invention will be described with reference to the flowchart shown in FIG. 14. The endoscope system 10 sets the observation mode to the special observation mode by a user operation with respect to the endoscope 11 or the processor device 13 (step ST110). By switching to the special observation mode, the endoscope 11 consecutively images the subject S with the pre-recognition imaging pattern in which the reference focus position and the moved focus position where the focus position is moved from the reference focus position are switched (step ST120).

In the pre-recognition imaging pattern, the processor device 13 acquires the endoscope image 70 captured by the endoscope 11 at the reference focus position and the focus position movement image captured by the endoscope 11 at the moved focus position (step ST130). The endoscope image 70 acquired by the processor device 13 is displayed on the screen, and image analysis is performed on the focus position movement image (step ST140). In a case in which the region of interest is not recognized in the image analysis (N in step ST150), the pre-recognition imaging pattern of acquiring the endoscope image 70 and the focus position movement image is continued (step ST130).

In a case in which the processor device 13 recognizes the region of interest in the image analysis (Y in step ST150), the processor device 13 acquires the focus position information of the focus position movement image in which the region of interest is recognized (step ST160). The imaging pattern is switched to the post-recognition imaging pattern, and the endoscope 11 performs imaging at the recognition-time focus position and the reference focus position based on the acquired focus position (step ST170).

In the post-recognition imaging pattern, the recognition-time focus position image and the endoscope image 70 are acquired, and the screen display and image analysis of the acquired recognition-time focus position image are performed (step ST180). After acquiring the recognition-time focus position image, in a case in which the observation in the special observation mode is continued (Y in step ST190), the observation is performed again with the pre-recognition imaging pattern according to the passage of time or the movement of the imaging range (step ST120). In a case in which the observation in the special observation mode is not continued (N in step ST190), the observation mode is switched to the normal observation mode, and the series of steps is ended. In a case in which the observation in the special observation mode is not continued, the endoscopic observation may be ended as it is instead of switching to the normal observation mode.

### Second Embodiment

In the special observation mode of the first embodiment, image analysis of the focus position movement image is performed in the pre-recognition imaging pattern, but in a second embodiment, image analysis is performed on the endoscope image 70. The focus position of the image to be used for the image analysis of the post-recognition imaging pattern is decided from the analysis result of the endoscope image 70. Other contents that are the same as those of the first embodiment will not be described.

In the pre-recognition imaging pattern in the second embodiment, the endoscope image 70 is captured in the same manner as in the normal observation mode. Image analysis is performed on the captured endoscope image 70 at an optional frequency. In a case in which the region of interest is not recognized through image analysis, the pre-recognition imaging pattern is continued, and in a case in which the region of interest is recognized, the focus position information to be applied to the post-recognition imaging pattern is estimated.

The image analysis unit 64 in the second embodiment has a function of estimating the focus position information appropriate for imaging the region of interest through image analysis. The focus position information may be estimated by the recognizer 65, which has trained using a data set including the focus position information, or the image analysis unit 64 may have an estimator (not shown) that is a trained model different from the recognizer 65.

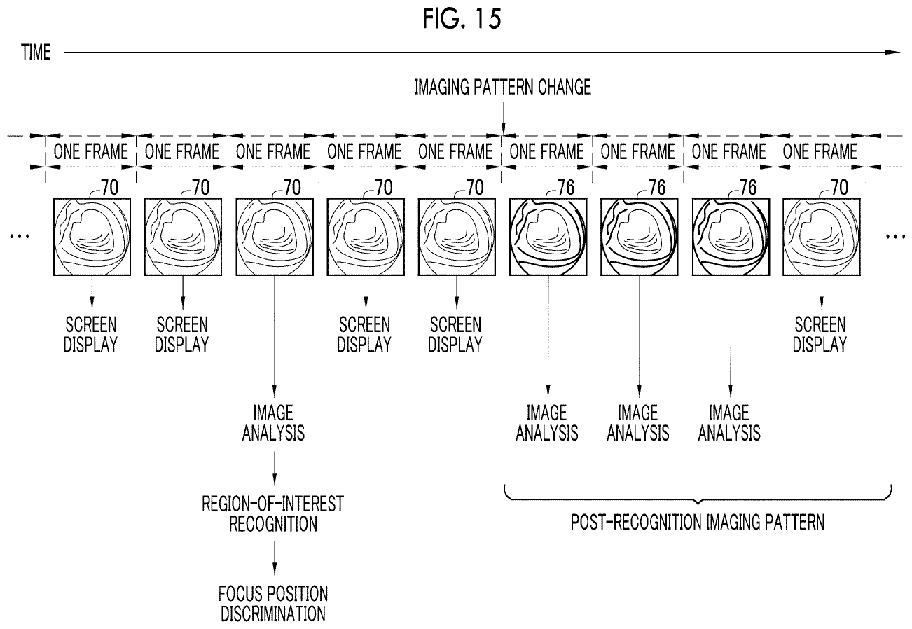

As shown in FIG. 15, in the pre-recognition imaging pattern, the endoscope images 70 are consecutively acquired, and the screen display and the image analysis of the endoscope image 70 are switched in a preset pattern in units of one frame. For example, image analysis of one frame is performed for every two frames of screen display. In a case in which the region of interest is recognized in the image analysis, the acquired focus position information is estimated, and the imaging pattern is switched to the post-recognition imaging pattern. In the post-recognition imaging pattern, an estimated focus position image 76 based on the estimated focus position is captured consecutively or at a higher frequency than the endoscope image 70. It is preferable that the estimated focus position image 76 is the same imaging pattern as the recognition-time focus position images in the first to third post-recognition imaging patterns of the first embodiment.

The estimated focus position image 76 applies to any image including the endoscope image 70, in addition to the near point image 72 and the far point image 74, because it is obtained at the focus position suitable for imaging the region of interest by estimation. In the special observation mode of the second embodiment, an appropriate focus position including the focus position of the endoscope image 70 is estimated, thereby enabling clear endoscopic observation and more accurate image analysis.

In the above embodiment, the hardware structure of a processing unit that executes various kinds of processing, such as the central controller, the light emission controller 22, the imaging controller 45, the image signal acquisition unit 50, the DSP 51, the noise reduction unit 52, the image processing unit 53, the output controller 54, and the special observation controller 60, is various processors as shown below. The various processors include a central processing unit (CPU) that is a general-purpose processor that executes software (programs) to function as various processing units, a graphical processing unit (GPU), a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be configured of one of these various processors, or may be configured of a combination of two or more processors of the same type or different types (for example, a plurality of FPGAs, a combination of a CPU and an FPGA, or a combination of a CPU and a GPU). In addition, a plurality of processing units may be configured of one processor. As an example in which the plurality of processing units are configured of one processor, first, as typified by computers such as a client or a server, one processor is configured of a combination of one or more CPUs and software, and this processor functions as the plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

Furthermore, the hardware structure of the various processors is more specifically an electric circuit (circuitry) having a form in which circuit elements such as semiconductor elements are combined. In addition, a hardware structure of a storage unit is a storage device such as a hard disc drive (HDD) or a solid state drive (SSD). In addition, from the above description, it is possible to grasp the endoscope system described in Appendices 1 to 10.

### Appendix 1

An endoscope system comprising:
an endoscope that images a subject; and
a processor,
in which the endoscope consecutively images the subject by switching between a reference focus position where a focus position is a preset position and a moved focus position where the focus position is moved from the reference focus position, and
the processor
   acquires an endoscope image captured by the endoscope at the reference focus position and a focus position movement image captured by the endoscope at the moved focus position,
   performs image analysis on the focus position movement image to recognize a region of interest from the subject, and
   displays the endoscope image on a screen and notifies of a result of the image analysis.

### Appendix 2

The endoscope system according to Appendix 1,
in which the endoscope captures, as the focus position movement image, at least one of a near point image in which the focus position is at a near point closer to the endoscope than in the endoscope image or a far point image in which the focus position is at a far point farther from the endoscope than in the endoscope image.

### Appendix 3

The endoscope system according to Appendix 2,
in which the processor
   measures a deviation amount of the focus position with respect to an in-focus position of the subject using a phase difference detection imaging element provided in the endoscope, and
   acquires at least one of position information of the near point or position information of the far point.

### Appendix 4

The endoscope system according to any one of Appendices 1 to 3,
in which the processor
   measures a movement amount of a distal end part of the endoscope,
   compares the movement amount with a predetermined reference range for the movement amount,
   reduces an imaging frequency of the focus position movement image with respect to the endoscope image in a case in which the movement amount is a value larger than the reference range, and
   increases the imaging frequency of the focus position movement image with respect to the endoscope image in a case in which the movement amount is a value smaller than the reference range.

### Appendix 5

The endoscope system according to Appendix 4,
in which, in a case in which the movement amount is a value included in the reference range, the endoscope fixes the imaging frequency of the focus position movement image with respect to the endoscope image and performs imaging.

### Appendix 6

The endoscope system according to any one of Appendices 1 to 5,
in which the endoscope captures a recognition-time focus position image based on the focus position at which the region of interest is recognized, at a higher imaging frequency than the endoscope image, and
the processor performs the image analysis on the recognition-time focus position image.

### Appendix 7

The endoscope system according to Appendix 6 or 7,
in which the processor displays the captured recognition-time focus position image on a screen.

### Appendix 8

The endoscope system according to Appendix 6,
in which the processor
   performs the image analysis on the endoscope image, and
   decides the recognition-time focus position image according to an analysis result of the endoscope image.

### Appendix 9

The endoscope system according to any one of Appendices 1 to 8,
in which the endoscope repeats an operation of capturing the endoscope image during a first period spanning a plurality of frames and capturing the focus position movement image during a second period spanning at least one frame.

### Appendix 10

The endoscope system according to any one of Appendices 1 to 9,
in which the processor controls an exposure amount of illumination light, which is emitted from a light source device connected to the endoscope and with which the subject is illuminated, according to the focus position.

### Explanation of References

10: endoscope system
11: endoscope
11a: insertion part
11b: operation part
11c: bendable part
11d: distal end part
11e: mode selector switch
11f: zoom operation portion
12: light source device
13: processor device
14: display
15: user interface
20: light source unit
22: light emission controller
29: light guide
30: illumination optical system
32: illumination lens
40: imaging optical system
42: objective lens
43: zoom lens
44: imaging sensor
44a: phase difference detection imaging element
44b: phase difference detection pixel
45: imaging controller
46: CDS/AGC circuit
48: A/D converter
50: image signal acquisition unit
51: DSP
52: noise reduction unit
53: image processing unit
54: output controller
60: special observation controller
62: analysis image acquisition unit
64: image analysis unit
65: recognizer
66: recognition result discrimination unit
68: imaging pattern change unit
70: endoscope image
72: near point image
74: far point image
76: estimated focus position image
R: imaging range
S: subject
T: focus target
ST110 to ST190: step

What is claimed is:

1. An endoscope system comprising:
an endoscope that images a subject; and
a processor,
wherein the endoscope consecutively images the subject by switching between a reference focus position where a focus position is a preset position and a moved focus position where the focus position is moved from the reference focus position, and
the processor is configured to
acquire an endoscope image captured by the endoscope at the reference focus position and a focus position movement image captured by the endoscope at the moved focus position,
perform image analysis on the focus position movement image to recognize a region of interest from the subject, and
display the endoscope image on a screen and notifies of a result of the image analysis.

2. The endoscope system according to claim 1,
wherein the endoscope captures, as the focus position movement image, at least one of a near point image in which the focus position is at a near point closer to the endoscope than in the endoscope image or a far point image in which the focus position is at a far point farther from the endoscope than in the endoscope image.

3. The endoscope system according to claim 2,
wherein the processor is configured to
measure a deviation amount of the focus position with respect to an in-focus position of the subject using a phase difference detection imaging element provided in the endoscope, and
acquire, based on the deviation amount, at least one of position information of the near point or position information of the far point.

4. The endoscope system according to claim 2,
wherein the processor is configured to
measure a movement amount of a distal end part of the endoscope,
compare the movement amount with a predetermined reference range for the movement amount,
reduce an imaging frequency of the focus position movement image with respect to the endoscope image in a case in which the movement amount is a value larger than the reference range, and
increase the imaging frequency of the focus position movement image with respect to the endoscope image in a case in which the movement amount is a value smaller than the reference range.

5. The endoscope system according to claim 4,
wherein, in a case in which the movement amount is a value included in the reference range, the endoscope fixes the imaging frequency of the focus position movement image with respect to the endoscope image and performs imaging.

6. The endoscope system according to claim 2,
wherein the endoscope captures a recognition-time focus position image based on the focus position at which the region of interest is recognized, at a higher imaging frequency than that of the endoscope image, and
the processor is configured to perform the image analysis on the recognition-time focus position image.

7. The endoscope system according to claim 6,
wherein the processor is configured to display the captured recognition-time focus position image on a screen.

8. The endoscope system according to claim 6,
wherein the processor is configured to
perform the image analysis on the endoscope image, and
decide the recognition-time focus position image according to an analysis result of the endoscope image.

9. The endoscope system according to claim 1,
wherein the endoscope repeats an operation of capturing the endoscope image during a first period spanning a plurality of frames and capturing the focus position movement image during a second period spanning at least one frame.

10. The endoscope system according to claim 1,
wherein the processor is configured to control an exposure amount of illumination light, which is emitted from a light source device connected to the endoscope and with which the subject is illuminated, according to the focus position.

11. A method of operating an endoscope system, the method comprising:
via an endoscope, consecutively imaging a subject by switching between a reference focus position, which is a preset focus position, and a moved focus position, which is the focus position moved from the reference focus position;
via a processor, acquiring an endoscope image captured by the endoscope at the reference focus position and a focus position movement image captured by the endoscope at the moved focus position;
via the processor, performing image analysis on the focus position movement image to recognize a region of interest from the subject; and
via the processor, displaying the endoscope image on a screen and notifying of a result of the image analysis.
